(19) United States
(12) Patent Application Publication
(10) Pub. No.: US 2010/0310459 A1
(43) Pub. Date: Dec. 9, 2010

(54) Targeted Detection of Dysplasia In Barrett's Esophagus With A Novel Fluorescence-Labeled Polypeptide

(75) Inventors: Thomas D. Wang, Ann Arbor, MI (US); Meng Li, Ann Arbor, MI (US)

Correspondence Address: MARSHALL, GERSTEIN & BORUN LLP, 233 SOUTH WACKER DRIVE, 6300 WILLIS TOWER, CHICAGO, IL, 60606-6357, US

(73) Assignee: THE REGENTS OF THE UNIVERSITY OF MICHIGAN, Ann Arbor, MI (US)

(21) Appl. No.: 12/763,033

(22) Filed: Apr. 19, 2010

Related U.S. Application Data
(60) Provisional application No. 61/170,614, filed on Apr. 18, 2009.
(60) Provisional application No. 61/302,388, filed on Feb. 8, 2010.

(51) Int. Cl. A61K 49/00; C07K 7/06; C07K 7/08

(52) U.S. Cl. 424/9.1; 530/329; 530/327

(57) ABSTRACT

The present invention is directed to compositions and methods for use in detecting dysplasia in Barrett's esophagus.

### CROSS-REFERENCE TO RELATED APPLICATIONS

This application claims the priority benefit under 35 U.S.C. §119(e) of U.S. Provisional Application No. 61/170,614, filed on Apr. 18, 2009, and U.S. Provisional Application No. 61/302,388, filed on Feb. 8, 2010, the disclosures of which are incorporated herein by reference in their entirety.

### STATEMENT OF GOVERNMENT INTEREST

This invention was made with government support under Grant Numbers R03 CA096752, CA136429, and CA093990, awarded by the National Institutes of Health (NIH). The government has certain rights in the invention.

### FIELD OF THE INVENTION

The present invention is directed to compositions and methods for use in detecting dysplasia in Barrett's esophagus.

### BACKGROUND OF THE INVENTION

Adenocarcinoma of the esophagus is growing at a rate faster than any other cancer in industrialized countries. This disease is a significant cause of morbidity and mortality, and Barrett's esophagus is a known precursor condition that results from a change in the lining of the esophageal mucosa and can be recognized endoscopically as salmon-colored mucosa that is confirmed histologically as intestinal metaplasia. The development of Barrett's mucosa is associated with central obesity related acid and bile reflux, and has an increased relative risk about 30 to 125 times higher for progression to cancer than that of normal esophagus. Barrett's mucosa transforms from normal tissue to cancer through a series of molecular and cellular changes. The histological classifications progress through stages that include squamous, intestinal metaplasia, low-grade dysplasia, high-grade dysplasia, and adenocarcinoma. Cancer can also develop directly from intestinal metaplasia at a rate of 1% per patient-year.

Conventional white light endoscopy is the most common method used for cancer screening in the setting of Barrett's esophagus [Wang et al., Am J. Gastroenterol. 2008; 103:788-97]. This modality employs sophisticated imaging optics to collect light over a very large field of view to rapidly survey the mucosal surface over areas with dimensions on the centimeter scale. This feature is necessary to scan the inner lining of hollow organs in the digestive tract in a practical time frame. However this approach has significant limitations. This technique relies on the reflection of white light from the tissue surface to reveal structural (anatomic) changes, and is not effective for the detection of flat dysplasia, such as that which occurs in the setting of Barrett's esophagus. Current screening and surveillance techniques are performed with medical endoscopy and are based on visualizing structural and morphological changes in the tissue that can be difficult to detect and interpret. Random fourquadrant biopsy is accepted as the standard of practice for screening, however, this approach is limited by a low yield for detection because dysplastic changes often occur in a spatially heterogeneous fashion [Levine et al., Gastroenterology 1993; 105:40-50].

The histological evaluation of excised esophageal mucosa for the presence of dysplasia is performed in a qualitative, subjective manner, and even among expert pathologists, substantial intra- and inter-observer variability in grading dysplasia occurs, limiting patient and physician confidence in the interpretation [Levine et al., Gastroenterology 1993; 105:40-50]. An incorrect evaluation of biopsy specimens may result in either an unnecessary esophagectomy or in progression to frank carcinoma. This lack of clarity in the prognostic value of conventional histopathology on the natural history of mucosa at risk for progression into adenocarcinoma demands the development of new criteria for pathological evaluation [Appelman, Arch Pathol Lab Med. 2005; 129:170-3]. In addition, new therapies aimed at inhibiting specific molecular targets have created widespread enthusiasm for drug discovery of anti-neoplastic agents that do not have systemic toxicities, and a greater understanding of the relationship between gene amplification and protein expression and the efficacy of targeted therapies is needed [Brabender et al., Cancer Epidemiol Biomarkers Prey 2005; 14:2113-7]. These factors combined with the poor survival rates associated with late stage adenocarcinoma provide significant motivation for further development of targeted, in vivo imaging strategies to improve screening of Barrett's mucosa, risk stratification of disease, and therapeutic options for cancer.

Given the prevalence of Barrett's esophagus in the general population, white light endoscopy with random biopsies has become the accepted method for cancer screening. However, this method is not effective for the detection of flat dysplasia, such as that which occurs in the setting of Barrett's esophagus. The histological evaluation of excised esophageal mucosa for the presence of dysplasia is performed in a qualitative, subjective manner, and even among expert pathologists, substantial intra- and inter-observer variability in grading dysplasia occurs, limiting patient and physician confidence in the interpretation. Therefore, because current methods of surveillance with white light endoscopy are non-specific and are limited by sampling error, improved imaging strategies are needed to localize pre-malignant mucosa for early detection and prevention of esophagus adenocarcinoma.

### SUMMARY OF THE INVENTION

Described herein are compositions and methods for detecting dysplasia in Barrett's esophagus. Accordingly, in one embodiment a composition is provided consisting of or consisting essentially of a polypeptide sequence as set out in SEQ ID NO: 1 (SNFYMPL), a linker sequence and a detectable marker; the detectable marker connected to the polypeptide through the linker, the linker having a net neutral charge, and wherein the presence of the linker results in an increase in detectable binding of the polypeptide sequence to Barrett's esophageal tissue compared to the detectable binding of the polypeptide sequence to Barrett's esophageal tissue in the absence of the linker.

In some aspects, a terminal amino acid of the linker is lysine. In further aspects, the linker comprises the sequence set out in SEQ ID NO: 2 (GGGSK).

In some embodiments, the detectable marker is fluorescein isothiocyanate (FITC).

The present disclosure also contemplates methods wherein compositions disclosed herein are administered to a human. Accordingly, in some embodiments a pharmaceutical composition is provided comprising a composition disclosed herein and a pharmaceutically acceptable excipient.

In some embodiments, compositions as provided herein further comprise an additional moiety, and in various aspects, the moiety is selected from the group consisting of a chemotherapeutic agent, a therapeutic agent, a polypeptide, an antibody, a nucleic acid, a small molecule or combinations thereof.

Further provided by the present disclosure is a method of detecting onset of a adenocarcinoma comprising the step of administering a composition disclosed herein in an amount effective to detect a Barrett's esophageal cell, the composition having a property of preferentially binding to the Barrett's esophageal cell relative to a non-cancerous cell thereby indicating the onset of adenocarcinoma cell development.

Additional aspects of the present disclosure provide a method of determining effectiveness of a treatment for dysplastic Barrett's esophagus in a human comprising the step of administering a composition of the present disclosure to the human in an amount effective to label dysplastic Barrett's esophagus, visualizing a first amount of cells labeled with a composition of the disclosure, and comparing the first amount to a previously visualized second amount of cells labeled with a composition of the disclosure, wherein a decrease in the first amount cells labeled relative to the previously visualized amount of cells labeled is indicative of effective treatment.

Further aspects of methods provided comprise obtaining a biopsy of the cell labeled by a composition of the present disclosure.

In some embodiments, the composition is administered after clinical onset of dysplastic Barrett's esophagus.

The present disclosure further provides a kit comprising a pharmaceutical composition of the disclosure, instructions for use of the composition and a device for administering the pharmaceutical composition to the patient.

### BRIEF DESCRIPTION OF THE FIGURES

FIG. 1a, Bound phage counting. ‘SNFYMPL’-phage and wild-type phage (no insert) were incubated with OE33 esophageal adenocarcinoma cells and Q-hTERT intestinal metaplasia cells, and the bound phage were recovered and titered. The number of ‘SNFYMPL’-phage that bound to the OE33 cells was about 1.6×10⁶ pfu compared to 6.5×10³ pfu for wild-type phage. On the other hand, the number of phage that bound to Q-hTERT cells were orders of magnitude less, 5.8×10⁴ and 1.7×10⁴ respectively (P<0.01). b, ELISA for Phage Binding Assay. The optical density on ELISA for binding of the ‘SNFYMPL’ phage to the OE33 esophageal adenocarcinoma cells is 1.23 compared with that of 0.72 and 0.73 for wild type phage and no phage (background), respectively. No difference in optical density on ELISA is observed when Q-hTERT cells were used (P<0.01).

FIG. 2 depicts a competitive inhibition study. The ‘SNFYMPL’ phage was incubated with the OE33 esophageal adenocarcinoma cells (1×10⁷), and the ‘SNFYMPL’ peptide and scrambled peptide ‘NLMPYFS’ were added to the cell-phage mixture at the concentration of 100, 200, and 400 μM to evaluate for competition. The bound phage were recoverd and tittered, and revealed that the ‘SNFYMPL’ significantly inhibited the SNFYMPL-phage binding to the OE33 cells in a concentration dependent manner. On the other hand, the scrambled peptide ‘NLMPYFS’ did not inhibit binding of the target peptide ‘SNFYMPL’ at any concentration (400 μM shown).

FIG. 3 shows fluoresence images of peptide binding to cell surface targets. The fluorescence-labeled peptide ‘SNFYMPL’ is seen binding to the plasma membrane in >90% of the OE33 (esophageal adenocarcinoma) cells but not on Q-hTERT (intestinal metaplasia) cells on the fluorescence image. The intensity associated with binding to the cell surface of OE33 was 69±18 compared to that of 25.7±2.5 for Q-hTERT. The DAPI stain reveals the extent of cell nuclei, and the overlay image shows that binding occurs on the cell surface.

### DETAILED DESCRIPTION OF THE INVENTION

Transformed cells and tissues express molecular changes well in advance of gross morphological features, thus providing a unique opportunity for the early detection of cancer. Greater sensitivity and specificity for disease-detection in the esophagus is achieved with the use of exogenous probes that target unique cancer expression molecular patterns. These probes are then labeled with detectable markers and detected on endoscopic imaging during routine screening to guide tissue biopsy for early detection of cancer, assess for sub-mucosal invasion, and monitor response to therapy.

Polypeptides have tremendous potential for clinical use as molecular probes to target molecular expression in vivo. In addition to high clonal diversity, small size, and compatibility with fluorescent dyes, polypeptides exhibit rapid binding kinetics and can be used clinically as a screening tool. Moreover, polypeptides can be topically administered to the luminal surface for binding to cell surface targets associated with pre-malignant (dysplastic) transformation with minimal concern for immunogenicity.

Described herein are compositions and methods for use in detecting dysplasia in Barrett's esophagus. The method utilizes labeled polypeptides as a molecular probe for in vivo detection of cancer in Barrett's esophagus patients combined with confocal fluorescence endoscopy.

Accordingly, in one embodiment a composition is provided consisting of or consisting essentially of a polypeptide sequence as set out in SEQ ID NO: 1, a linker sequence and a detectable marker; the detectable marker connected to the polypeptide through the linker, the linker having a net neutral charge, and wherein the presence of the linker results in an increase in detectable binding of the polypeptide sequence to Barrett's esophageal tissue compared to the detectable binding of the polypeptide sequence to Barrett's esophageal tissue in the absence of the linker.

In some aspects, the detectable binding takes place in vivo. In some aspects, the detectable binding takes places in vitro. In still further aspects, the detectable binding takes place in situ. In situ means “in the natural or normal place.” For example and without limitation, examining a cell within a whole organ intact and under perfusion is an in situ investigation. This would not be in vivo as the organ has been removed from the organism, but it would not be the same as working with the cell alone (a common scenario in in vitro experiments). One of ordinary skill in the art will understand that the compositions of the present disclosure are useful, for example and without limitation, in each of the aforementioned applications.

Certain methods of the invention are those wherein effectiveness of a treatment for dysplastic Barrett's esophagus in a human is determined comprising the step of administering a composition of the disclosure to the human in an amount effective to label dysplastic Barrett's esophagus, visualizing a first amount of cells labeled with the composition, and comparing the first amount to a previously visualized second amount of cells labeled with the composition, wherein a decrease in the first amount amount cells labeled relative to the previously visualized amount of cells labeled is indicative of effective treatment. In these aspects, a decrease of 5% is indicative of effective treatment. In other aspects, a decrease of about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or more is indicative of effective treatment.

### Linkers

As used herein, a “linker” is a sequence of uncharged amino acids located at a terminus of a polypeptide of the disclosure. The presence of a linker has been found to result in an increase in detectable binding of a polypeptide sequence to Barrett's esophageal tissue compared to the detectable binding of the polypeptide sequence to Barrett's esophageal tissue in the absence of the linker. In some embodiments, the linker sequence terminates with a lysine residue. In various aspects, the detectable marker as described herein is attached to the linker. In further aspects, the linker sequence is GGGSK (SEQ ID NO: 2). In further aspects, the detectable marker is FITC. Uncharged amino acids contemplated by the present disclosure include but are not limited to glycine, serine, cysteine, threonine, histidine, tyrosine, asparagine, and glutamine.

In some aspects, the presence of a linker results in at least a 1% increase in detectable binding of a polypeptide sequence to Barrett's esophageal tissue compared to the detectable binding of the polypeptide sequence to Barrett's esophageal tissue in the absence of the linker. In various aspects, the increase in detectable binding of a polypeptide sequence to Barrett's esophageal tissue compared to the detectable binding of the polypeptide sequence to Barrett's esophageal tissue in the absence of the linker is at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 100-fold or more.

### Polypeptides

The term “polypeptide” refers to molecules of 2 to 50 amino acids, molecules of 3 to 20 amino acids, and those of 6 to 15 amino acids. In one aspect, polypeptides and linkers as contemplated by the invention are 5 amino acids in length. In various aspects, a polypeptide or linker are 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length.

Exemplary polypeptides may be randomly generated by methods known in the art, carried in a polypeptide library (for example and without limitation, a phage display library), derived by digestion of proteins, or chemically synthesized. Polypeptides of the present disclosure have been developed using techniques of phage display, a powerful combinatorial method that uses recombinant DNA technology to generate a complex library of polypeptides for selection by preferential binding to cell surface targets [Scott et al., Science 1990; 249:386-90.]. The protein coat of bacteriophage, such as the filamentous M13 or icosahedral T7, is genetically engineered to express a very large number (>10⁹) of different polypeptides with unique sequences to achieve affinity binding [Cwirla et al., PNAS 1990; 87:6378-82]. Selection is then performed by biopanning the phage library against cultured cells and tissues that over express the target. The DNA sequences of these candidate phage are then recovered and used to synthesize the polypeptide [Pasqualini et al., Nature 1996; 380:364-6].

Polypeptides include D and L form, either purified or in a mixture of the two forms. Also contemplated by the present disclosure are polypeptides that compete with polypeptides of the disclosure for binding to Barrett's esophageal tissue.

In specific aspects, the present disclosure provides two seven residue polypeptides (7-mers) and one twelve residue polypeptide (12-mer) identified using techniques of phage display by biopanning against OE33 human esophageal adenocarcinoma cell lines in culture. These polypeptide sequences are 1) SNFYMPL (SEQ ID NO: 1); 2) VATQAYL (SEQ ID NO: 3), and 3) GLKIWSLPPHHG (SEQ ID NO: 4). In other aspects, a polypeptide is provided that is at least 80% identical to the polypeptides disclosed herein. In further aspects, a polypeptide is provided that is at least 85%, 90%, 95%, or at least 99% identical to the polypeptides disclosed herein. It will be understood and appreciated by those of ordinary skill in the art that additional polypeptides may be identified using phage display and utilized in the compositions and methods of the present disclosure.

In one aspect, the polypeptide sequence ASYNYDA (SEQ ID NO: 5) is contemplated by the present disclosure.

It will be understood that polypeptides and linkers of the invention may incorporate modifications known in the art and that the location and number of such modifications may be varied to achieve an optimal effect.

### Detectable Markers

As used herein, a “detectable marker” is any label that can be used to identify the binding of a composition of the disclosure to esophageal tissue. Non-limiting examples of detectable markers are fluorophores, chemical or protein tags that enable the visualization of a polypeptide. Visualization may be done with the naked eye, or a device (for example and without limitation, an endoscope) and may also involve an alternate light or energy source.

Fluorophores, chemical and protein tags that are contemplated for use in the methods of the invention include but are not limited to FITC, Cy 5.5, Cy 7, Li—Cor, a radiolabel, biotin, luciferase, 1,8-ANS (1-Anilinonaphthalene-8-sulfonic acid), 1-Anilinonaphthalene-8-sulfonic acid (1,8-ANS), 5-(and -6)-Carboxy-2′,7′-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA pH 7.0, 5-TAMRA-MeOH, 6 JOE, 6,8-Difluoro-7-hydroxy-4-methylcoumarin pH 9.0, 6-Carboxyrhodamine 6G pH 7.0, 6-Carboxyrhodamine 6G, hydrochloride, 6-HEX, SE pH 9.0, 6-TET, SE pH 9.0, 7-Amino-4-methylcoumarin pH 7.0, 7-Hydroxy-4-methylcoumarin, 7-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody conjugate pH 7.2, Alexa Fluor 488 antibody conjugate pH 8.0, Alexa Fluor 488 hydrazide-water, Alexa Fluor 532 antibody conjugate pH 7.2, Alexa Fluor 555 antibody conjugate pH 7.2, Alexa Fluor 568 antibody conjugate pH 7.2, Alexa Fluor 610 R-phycoerythrin streptavidin pH 7.2, Alexa Fluor 647 antibody conjugate pH 7.2, Alexa Fluor 647 R-phycoerythrin streptavidin pH 7.2, Alexa Fluor 660 antibody conjugate pH 7.2, Alexa Fluor 680 antibody conjugate pH 7.2, Alexa Fluor 700 antibody conjugate pH 7.2, Allophycocyanin pH 7.5, AMCA conjugate, Amino Coumarin, APC (allophycocyanin) Atto 647, BCECF pH 5.5, BCECF pH 9.0, BFP (Blue Fluorescent Protein), Calcein, Calcein pH 9.0, Calcium Crimson, Calcium Crimson Ca2+, Calcium Green, Calcium Green-1 Ca2+, Calcium Orange, Calcium Orange Calif. 2+, Carboxynaphthofluorescein pH 10.0, Cascade Blue, Cascade Blue BSA pH 7.0, Cascade Yellow, Cascade Yellow antibody conjugate pH 8.0, CFDA, CFP (Cyan Fluorescent Protein), CI-NERF pH 2.5, CI-NERF pH 6.0, Citrine, Coumarin, Cy 2, Cy 3, Cy 3.5, Cy 5, CyQUANT GR-DNA, Dansyl Cadaverine, Dansyl Cadaverine, MeOH, DAPI, DAPI-DNA, Dapoxyl (2-aminoethyl) sulfonamide, DDAO pH 9.0, Di-8 ANEPPS, Di-8-ANEPPS-lipid, DiI, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody conjugate pH 8.0, Erythrosin-5-isothiocyanate pH 9.0, eYFP (Enhanced Yellow Fluorescent Protein), FDA, FITC antibody conjugate pH 8.0, FlAsH, Fluo-3, Fluo-3 Ca2⁺, Fluo-4, Fluor-Ruby, Fluorescein, Fluorescein 0.1 M NaOH, Fluorescein antibody conjugate pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca2⁺, Fura Red, high Ca, Fura Red, low Ca, Fura-2 Ca2+, Fura-2, Fura-2, GFP (S65T), HcRed, Indo-1 Ca2⁺, Indo-1, Ca free, Indo-1, Ca saturated, JC-1, JC-1 pH 8.2, Lissamine rhodamine, Lucifer Yellow, CH, Magnesium Green, Magnesium Green Mg2+, Magnesium Orange, Marina Blue, mBanana, mCherry, mHoneydew, mOrange, mPlum, mRFP, mStrawberry, mTangerine, NBD-X, NBD-X, MeOH, NeuroTrace 500/525, green fluorescent Niss1 stain-RNA, Nile Blue, Nile Red, Nile Red-lipid, Nissl, Oregon Green 488, Oregon Green 488 antibody conjugate pH 8.0, Oregon Green 514, Oregon Green 514 antibody conjugate pH 8.0, Pacific Blue, Pacific Blue antibody conjugate pH 8.0, Phycoerythrin, R-Phycoerythrin pH 7.5, ReAsH, Resorufin, Resorufin pH 9.0, Rhod-2, Rhod-2 Ca2⁺, Rhodamine, Rhodamine 110, Rhodamine 110 pH 7.0, Rhodamine 123, MeOH, Rhodamine Green, Rhodamine phalloidin pH 7.0, Rhodamine Red-X antibody conjugate pH 8.0, Rhodamine Green pH 7.0, Rhodol Green antibody conjugate pH 8.0, Sapphire, SBFI-Na⁺, Sodium Green Na⁺, Sulforhodamine 101, Tetramethylrhodamine antibody conjugate pH 8.0, Tetramethylrhodamine dextran pH 7.0, and Texas Red-X antibody conjugate pH 7.2.

Non-limiting examples of chemical tags contemplated by the present disclosure include radiolabels. For example and without limitation, radiolabels that may be used in the compositions and methods of the present disclosure include ¹¹C, ¹³N, ¹⁵O, ¹⁸F, ³²P, ⁵²Fe, ⁶²Cu, ⁶⁴Cu, ⁶⁷Cu, ⁶⁸Ga, ⁸⁶Y, ⁸⁹Zr, ⁹⁰Y, ⁹⁴mTc, ⁹⁴Tc, ⁹⁵Tc, ⁹⁹mTc, ¹⁰³pd, ¹⁰⁵Rh, ¹⁰⁹Pd, ¹¹¹Ag, ¹¹¹In, ¹²³I, ¹²⁴I, ¹²⁵I, ¹³¹I, ¹⁴⁰La, ¹⁴⁹Pm, ¹⁵³Sm, ¹⁵⁴⁻¹⁵⁹Gd, ¹⁶⁵Dy, ¹⁶⁶Dy, ¹⁶⁹Yb, ¹⁷⁵Yb, ¹⁷⁵Lu, ¹⁷⁷Lu, ¹⁸⁶Re, ¹⁸⁸Re, ¹⁹²Ir, ¹⁹⁸Au, ¹⁹⁹Au, and ²¹²Bi.

A worker of ordinary skill in the art will appreciate that there are many such detectable markers that can be used to visualize a composition of the disclosure, either in vitro or in vivo.

### Additional Moieties

In another embodiment, a composition is provided further comprising an additional moiety, said composition having the property of detecting a adenocarcinoma cell. In various aspects and without limitation, the additional moiety is a polypeptide, a small molecule, a therapeutic agent, a chemotherapeutic agent, or combinations thereof.

The term “small molecule”, as used herein, refers to a chemical compound, for instance a peptidometic or oligonucleotide that may optionally be derivatized, or any other low molecular weight organic compound, either natural or synthetic.

By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, about 1000 or more Daltons.

In some aspects, the additional moiety is a protein therapeutic. Protein therapeutic agents include, without limitation, cellular or circulating proteins as well as fragments and derivatives thereof. Still other therapeutic agents include polynucleotides, including without limitation, protein coding polynucleotides, polynucleotides encoding regulatory polynucleotides, and/or polynucleotides which are regulatory in themselves. Optionally, the compositions may comprise a combination of the compounds described herein.

In various aspects, protein therapeutic agents include cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor al, glial cell line-derived neutrophic factor receptor a2, growth related protein, growth related protein α, growth related protein β, growth related protein y, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor receptor, TNF, including TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

Therapeutic agents also include, as one specific embodiment, chemotherapeutic agents. A chemotherapeutic agent contemplated for use in a composition of the invention includes, without limitation, alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinum coordination complexes such as cisplatin and carboplatin, anthracenediones such as mitoxantrone, substituted urea such as hydroxyurea, methylhydrazine derivatives including N-methylhydrazine (M1H) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

Dosages of the therapeutic can be administered as a dose measured in mg/kg. Contemplated mg/kg doses of the disclosed therapeutics include about 1 mg/kg to about 60 mg/kg. Specific ranges of doses in mg/kg include about 1 mg/kg to about 20 mg/kg, about 5 mg/kg to about 20 mg/kg, about 10 mg/kg to about 20 mg/kg, about 25 mg/kg to about 50 mg/kg, and about 30 mg/kg to about 60 mg/kg. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

“Effective amount” as used herein refers to an amount of a detectably labeled polypeptide sufficient to visualize the identified disease or condition, or to exhibit a detectable therapeutic or inhibitory effect. The effect can be detected by, for example, an improvement in clinical condition or reduction in symptoms. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

### Visualization of Compositions

Visualization of binding to targeted Barrett's esophageal tissue can be by any means known to those of ordinary skill in the art. As discussed herein, visualization can be, for example and without limitation, in vivo, in vitro, or in situ visualization. “Visualization” and “detection” are used interchangeably herein.

In one embodiment, visualization is performed via imaging and may be performed with a wide area endoscope (Olympus Corporation, Tokyo, Japan) that is designed specifically to collect fluorescence images with high spatial resolution over large mucosal surface areas on the macroscopic scale (millimeters to centimeters). This capability is needed to rapidly screen large surface areas such as that found in the distal esophagus during endoscopy to localize regions suspicious for disease [Wang et al., Gastrointestinal Endoscopy 1999; 49:447-55]. This technique has been adapted for fluorescence detection, and is compatible with dye-labeled probes. This instrument can image in three different modes, including white light (WL), narrowband imaging (NBI), and fluorescence imaging. Narrow-band imaging is a new technology that represents a variation of conventional white light illumination by altering the spectrum with optical filters to restrict or narrow the range of wavelengths.

The method enhances contrast in the endoscopic images to provide more visual details of the esophageal mucosa by tuning the light to maximize absorption of hemoglobin present in the vasculature of regions of intestinal metaplasia. The WL and NBI images are collected by the central objective lens, and the fluorescence image is collected by a second objective lens located near the periphery. There is a distance of approximately 3 mm between the centers of the white light and fluorescence objectives that results in only a slight misregistration of the two images. Furthermore, there is an air/water nozzle that removes debris from the objective lenses, and a 2.8 mm diameter instrument channel that can be used to deliver biopsy forceps. The objectives are forward viewing and have a field of view (FOV), defined by maximum angle of illumination, of 140 deg. The WL/NBI imaging modes have a depth of field (DOF), defined by range of distances between the distal end of the endoscope to the mucosal surface whereby the image is in focus, of 7 to 100 mm, and that for fluorescence is 5 to 100 mm. The transverse resolution measured at a distance of 10 mm from the mucosa for WL/NBI is 15 μm and for fluorescence is 20 μm. A xenon light source provides the illumination for all three modes, which is determined by a filter wheel located in the image processor. Illumination for all three modes of imaging is delivered through the two fiber light guides. In the WL mode, the full visible spectrum (400 to 700 nm) is provided, while in the NBI mode, a filter wheel narrows the spectral bands in the red, green, and blue regime. In the fluorescence mode, a second filter wheel enters the illumination path, and provides fluorescence excitation in the 395 to 475 nm spectral band. In addition, illumination from 525 to 575 nm provides reflected light in the green spectral regime centered at 550 nm. The fluorescence image is collected by the peripherally located CCD detector that has a 490-625 nm band pass filter for blocking the excitation light. Normal mucosa emits bright autofluorescence, thus the composite color appears as bright green. Because the increased vasculature in neoplastic mucosa absorbs autofluorescence, it appears with decreased intensity.

This medical endoscope can be used to collect images after polypeptide administration and incubation from Barrett's esophagus with known dysplastic changes with 1) white light, 2) narrow band, and fluorescence. After entering the distal esophagus, a 5 second video is collected and digitized in the white light and narrow band imaging modes. The imaging in this mode is used to assess the spatial extent of the intestinal metaplasia for comprehensive evaluation of polypeptide binding. Then, approximately 3 ml of the fluorescence-labeled polypeptide is administered topically at a concentration of 10 μM to the distal esophagus using a mist spray catheter being careful to cover the full extent of the metaplastic mucosa. Amounts of fluorescently-labeled polypeptide can be determined by one of ordinary skill in the art.

Routine endoscopic examination of the stomach, including the antrum, fundus, cardia, and incisura, and the first and second portions of the duodenum is then performed, allowing for the polypeptide to incubate for a total of 5 minutes. The endoscope is then retracted back to the distal esophagus where the unbound polypeptides were gently rinsed off with water, and another 10 second video was collected of the polypeptide targeted fluorescence image. The mucosa of the distal esophagus is then removed en bloc by techniques of endoscopic mucosal resection (EMR), and sent for histological evaluation.

In some embodiments, the detectable label is a radiolabel that is detected by, in some aspects, nuclear imaging. Nuclear imaging is understood in the art to be a method of producing images by detecting radiation from different parts of the body after a radioactive tracer material is administered. The images are recorded on computer and on film.

Other methods of the invention involve the acquisition of a tissue sample from a patient. The tissue sample is selected from the group consisting of a tissue or organ of said patient.

### Formulations

In an embodiment, the pharmaceutical compositions may be formulated with pharmaceutically acceptable excipients such as carriers, solvents, stabilizers, adjuvants, diluents, etc., depending upon the particular mode of administration and dosage form. The pharmaceutical compositions are generally formulated to achieve a physiologically compatible pH, and range from a pH of about 3 to a pH of about 11, about pH 3 to about pH 7, depending on the formulation and route of administration. In alternative embodiments, the pH is adjusted to a range from about pH 5.0 to about pH 8. In various aspects, the pharmaceutical compositions comprise a therapeutically effective amount of at least one compound as described herein, together with one or more pharmaceutically acceptable excipients. Optionally, the pharmaceutical compositions comprises a combination of the compounds described herein, or may include a second active ingredient useful in the treatment or prevention of bacterial growth (for example and without limitation, anti-bacterial or anti-microbial agents), or may include a combination of polypeptides of the invention.

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol) wetting or emulsifying agents, pH buffering substances, and the like.

The invention will be more fully understood by reference to the following example which details exemplary embodiments of the invention. It should not, however, be construed as limiting the scope of the invention. All citations throughout the disclosure are hereby expressly incorporated by reference.

### EXAMPLES

### Example 1

Pepide Selection. Peptide selection was performed using techniques of phage display (Ph.D.-7, New England Biolabs, Beverly, Mass.). The esophageal adenocarcinoma cell line OE33 was maintained in RPMI-1640 media supplemented with 10% FBS. The Barrett's esophagus (intestinal metaplasia) cell lines KR-42421 (Q-hTERT, non-dysplastic) was maintained in keratinocyte-serum free medium supplemented with bovine pituitary extract (BPE) and human recombinant epidermal growth factor (rEGF) (Invitrogen, Carlsbad, Calif.). All cell lines were incubated at 37° C. in 5% CO₂.

Biopanning was carried out by using a subtractive whole-cell approach. Q-hTERT cells in log-phase growth were detached with cell dissociation buffer (Invitrogen, Carlsbad, Calif.) and blocked with blocking buffer (PBS with 1% bovine serum albumin) for 45 minutes on ice. Ten μl of Ph.D.-7 random phage library (1.5×10″ plaque-forming unit) was suspended in 5 ml PBS and biopanned with 1.0×10⁷ Q-hTERT cells for 30 min at room temperature (RT). The cells were spun down in a centrifuge at 1000 rpm for 6 minutes and the supernatant containing unbound phage was transferred to another 1.0×10⁷ Q-hTERT cells for a second round of clearance. The resulting supernatant was then amplified, precipitated with PEG-NaCl, and then titered according to the manufacturer's instructions.

For the enrichment of phage that bound to the OE33 esophageal andenocarcinoma cells, 1×10¹¹ pfu phage from the former step was added to 1.0×10⁶ OE33 cells which were detached and blocked as described above. After ˜30 minutes of gentle agitation at room temperature, the cells were spun down and the supernatant with unbound phage was discarded. The cells were washed with PBS/0.1% (v/v) Tween-20 a total of 10 times. The bound phages were then eluted with 1 ml of 0.2 M glycine, pH 2.2/0.1% BSA for 8 min. The phage-containing solution was immediately neutralized with 150 μL of 1 M Tris, pH 9.5. After amplification, the same amount of phage were panned against the OE33 cells for another round with same protocol except 2 min elute buffer (0.2 M glycine, pH 2.2/0.1% BSA) washing before elution. Another two rounds panning of a total four rounds were carried out following the same protocol.

Sixty single phage plaques from the last round panning were selected randomly, amplified individually and sequenced (DNA Sequencing Core, University of Michigan, Mich.). Result peptides sequences were analyzed by the National Center of Biotechnology Information BLAST search using the option for short nearly exact matches, to identify human proteins with homologous sequences.

After sequencing this pool of phage, 49 clones were found to have the same peptide sequence ‘SNFYMPL’. The other 11 clones expressed different peptide sequences that appeared only once each.

### Example 2

Phage Binding Assay. OE33 and Q-hTERT cells were detached and blocked as described above. The candidate SNFYMPL-phage or control phage (randomly insert) were incubated with OE33 cells (1×10⁷) or Q-hTERT cells (1×10⁷) for 30 min with gentle agitation at room temperature. After washing ten times with PBS/0.1% (v/v) Tween-20 and one time with 0.2 M glycine, pH 2.2/0.1% BSA for two minutes, the bound phages were recovered and titered. Every sample was carried out in triplicate. The amount of bound phages in every sample was calculated using student's t test.

Cell ELISA for Phage Binding Assay. OE33 cells and Q-hTERT cells were grown to 100% confluence in a 96-well plate and incubated sequentially with 2×10⁷ pfu SNFYMPL-phage or control phage for 10 minutes in triplicate at room temperature, washed with PBS containing 0.1% Tween-20 six times, incubated with HRP-labeled anti-M13 antibody (Fitzgerald, Concord, Mass.), developed with TMB (Invitrogen, Carlsbad, Calif.), and absorbance 650 nm was determined (Emax, Molecular Devices).

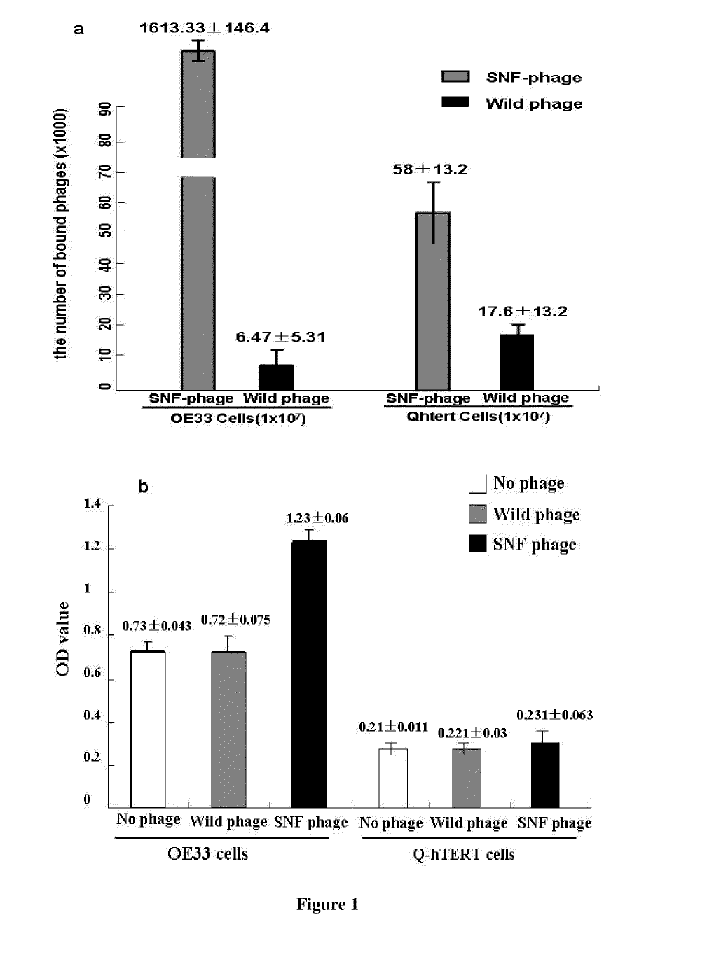

Pepide Validation. From the results of bound phage quantity, SNFYMPL-phage number was about 250 times higher than control phage (randomly insert) when using OE33 cells to test. This ratio dramatically decreased to 3 when the phages were apllied on Q-hTERT cells (p<0.01). (FIG. 1a). On ELISA assay, the optical density for binding of the SNFYMPL-phage to OE33 cells is almost a factor of two greater than that compared to wild type phage and no phage (background control). A much lower OD was observed for binding to Q-hTERT cells (FIG. 1b).

Peptide synthesis. The targeted peptide sequence ‘SNFYMPL’ identified by the phage binding assay was synthesized using standard (F)luorenyl-(m)eth(o)xy-(c)arbonyl (FMOC) chemistry, purified to a minimum of 90% purity using high-performance liquid hromatography (HPLC), and analyzed by reverse phase HPLC and mass spectrometry. The fluorescence dye FITC was conjugated at the C-terminus of the peptide via a flexible 5-amino acid linker (SNFYMPL-GGGSK-FITC; SEQ ID NO: 6). GGGSK is the same linker as this peptide is fused to the coat protein pIII of M13. The targeted peptide was scrambled to form the sequence ‘NLMPYFS-GGGSK’ (SEQ ID NO: 7) and synthesized as described above for use as a control.

Competitive inhibition assay. The OE33 and Q-hTERT cells were detached and blocked. 2×10″ pfu of SNFYMPL-phage was incubated with OE33 cells (1×10⁷) or Q-hTERT cells (1×10⁷), ‘SNFYMPL-GGGSK’ peptide or scrambled peptide ‘NLMPYFS-GGGSK’ were added into cell-phage mixture at the concentration of 100, 200, and 400 μm for competition with the phage. After washing three times with PBS/0.1% (v/v) Tween-20 and one time with 0.2 M glycine, pH 2.2/0.1% BSA for two minutes, the bound phage were recovered and titered. Every sample was carried out in triplicate. The amount of bound phage in every sample was calculated using a student's t test.

The competitive inhibition between FITC-labeled peptide and unlabeled peptide was done by incubating the unlabeled peptide with OE33 cells for 15 minutes prior to add the FITC-labeled peptide (100 μm) in three different concentrations, 100, 500, and 1000 μm. Three fluorescence images were collected at 200× from each well of the chamber slide using the same gain and exposure time. Images selected for analysis met the following criteria: 1) 70-90% cell confluence, 2) away from the edge of each well, 3) low background binding on cell free area of slide, 4) No change in cellular morphology. The mean cell numbers under three 200× views were record to calculate the percentage of peptide binding cells. Quantification of the fluorescence intensities was done by NIH Image J software to calculate pixel value between each group under the same threshold. Differences in the mean fluorescence intensities were c edge ompared using a student's t test.

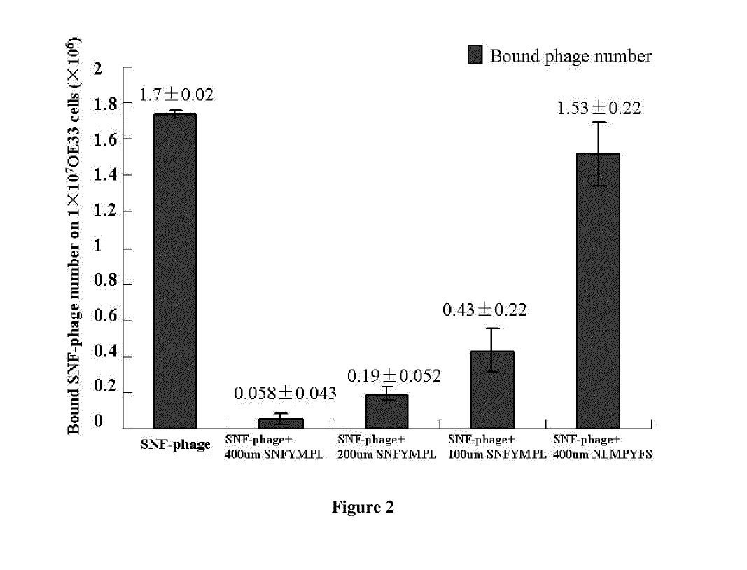

For further proof that the SNFYMPL-phage OE33 cells binding ability depended on insert peptide but not other phage coat protein absorption, ‘SNFYMPL-GGGSK’ peptide were used for competition with SNFYMPL-phage on OE33 cells. Scrambled peptide ‘NLMPYFS-GGGSK’ was used as control. ‘SNFYMPL-GGGSK’ could obviously inhibit the SNFYMPL-phage bound with OE33 cells and this ability was concentration dependent. Scrambled peptide, ‘NLMPYFS-GGGSK’, had no inhibition ability against SNFYMPL-phage (FIG. 2), which demonstrated that the SNFYMPL-phage binding ability come from insert peptide and this was a specific binding.

### Example 3

Fluoresence images for peptide binding on culture cells. The OE33 and QhTERT cells were grown in chamber slides to 80% confluence. Blocking of non-specific binding to these cells was performed by adding 200 μl of 1% BSA diluted in PBS for 30 min. The cells were then incubated with 100 μmol of the candidate FITC-labeled peptide in serum free media for 10 minutes at room temperature. The cells were washed 3 times using 200 μL PBS/0.5% TWEEN 20 in room temperature. The cells were fixed in ice cold 4% praformaldehyde for 5 minutes. The cells were then stained with Vectashield mounting medium containing DAPI. Fluorescence images were collected with a confocal microscope (Nikon 1000) at 200×. The fluorescence intensity from the cells in 3 images was averaged to assess for peptide binding using NIH Image J software.

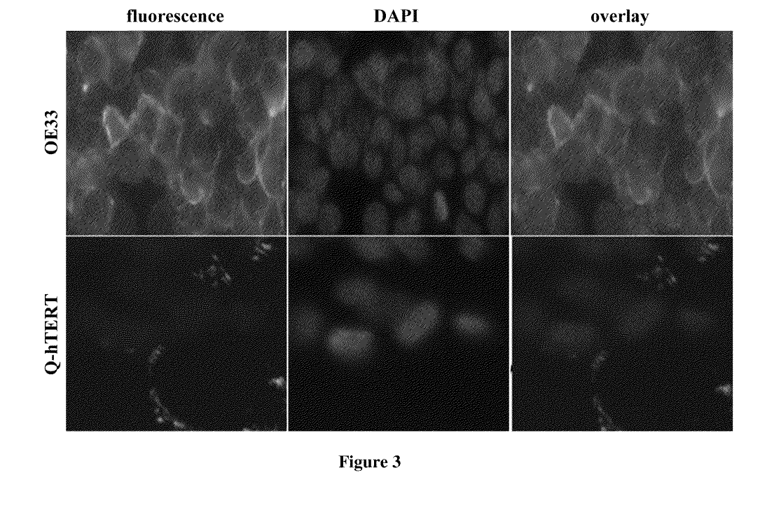

Under the fluoresence microscope, SNFYMPL-GGGSK-FITC was bound to the plasma membrane of >90% OE33 cells but not on Q-hTERT (normal Barrett's) cells (FIG. 3). The intensity associated with binding to the cell surface of OE33 was 25.738±2.504 (mean grey value) compared to that of 69.033±18.007 (mean grey value) for Q-hTERT using NIH Image J software analysis.

The SNFYMPL peptide has been shown herein to have high affinity and specificity for diseased tissues, and can be detected on endoscopic imaging to help guide tissue biopsy and increase the yield of detection of pre-malignant mucosa.

### Example 4

Identification of plasma membrane targets on adenocarcinoma cells that affinity bind to “ASYNYDA” (SEQ ID NO: 5). The carboxyl end of the target peptide was linked to affinity column beads using EDC chemistry. Seg-1 cells were grown to confluency, lysed by sonification, and fractionated by centrifugation. The resulting membrane fractions were applied to the target peptide labeled column and specific binding proteins were eluted. These proteins were separated and collected in fractions by a reverse phase column using a Beckman PF 2D liquid chromatography apparatus. The protein fractions were spotted on a nitrocellulose coated micro array printer slide and probed with the FITC-labeled target peptide. The fluorescence intensities at each spot were quantified with an Axon Imager. Protein fractions with the highest intensity were trypsinized, and proteomic analysis was performed using a Finnigan LTQ linear ion trap mass spectrometer. The protein identities were confirmed using an International Protein Index (IPI) database search, utilizing the SEQUEST program and with open source Xtandem and peptide and protein prophet software. The target peptide “ASYNYDA” (100 μM) (SEQ ID NO: 5) showed preferential binding to cell surface targets on SEG1 (adenocarcinoma) and lack of binding to 0E-21 (squamous) and Q-hTERT (intestinal metaplasia) cells. Cell surface targets included Annexin A2, Hepatoma-derived growth factor, Histone H₂B, Histone H2A, and Junction plakoglobin. See Table 1 below for list of targets identified. All identified proteins were either an exact match or >0.99. Although some of the proteins are normally found in the nucleus, there is evidence to show that these proteins translocate to the plasma membrane in cancer cells, most notably the histones.

TABLE 1

| Fraction | Probability | Protein Description |
|---|---|---|
| 1 | 1.0000 | Gene_Symbol = HIST1H2BN Histone H2B type 1-N |
| | 1.0000 | Gene_Symbol = THOC4 THO complex subunit 4 |
| | 1.0000 | Gene_Symbol = LOC388177 similar to Histone H2AV |
| | 1.0000 | Gene_Symbol = HMGA1 HMGA1 protein |
| | 0.9992 | Gene_Symbol = RPL7A Ribosomal protein L7a |
| | 0.9920 | Gene_Symbol = HMGN1 Uncharacterized protein HMGN1 |
| | 0.9782 | Gene_Symbol = ANXA2 Annexin A2 |
| | 0.9462 | Gene_Symbol = HMGB1 High-mobility group box 1 |
| | 0.9292 | Gene_Symbol = HDGF Hepatoma-derived growth factor |
| 9 | 1.0000 | Gene_Symbol = HIST1H2BK Histone H2B type 1-K |
| 11 | 1.0000 | Gene_Symbol = UBB; RPS27A; UBC 16 kDa protein |
| 24 | 1.0000 | Gene_Symbol = MGC102966 20 kDa protein |
| | 0.9989 | Gene_Symbol = JUP Junction plakoglobin |
| 25 | 1.0000 | Gene_Symbol = H2AFX Histone H2A.x |
| | 1.0000 | Gene_Symbol = HIST1H2AC Histone H2A type 1-C |
| | 1.0000 | Gene_Symbol = HIST3H2A Histone H2A type 3 |

### Example 5

### Validation of Preferential Binding of the Fluorescence-Labeled SNF Polypeptide to Barrett's Dysplasia on the Surface of Esophageal Mucosa

Human endoscopic mucosal resection (EMR) specimens were freshly taken from regions of suspected dysplastic mucosa. Each specimen was incubated with the FITC-labeled polypeptide of SEQ ID NO: 1 at a concentration of 100 μm for 5 minutes. Ink was used to mark the 12 o'clock position for orientation. White light stereo microscope (Olympus SZX16) images were obtained with an overlying 20×20 mm grid to register the histology. Fluorescence images were obtained at an exposure of 12 ms. The tissue was then fixed in formalin. An expert GI pathologist sectioned the specimens along cross-sections at ˜2 mm intervals longitudinally and interpreted the histopathology while blinded to the fluorescence images. The mean intensity of the fluorescence images in each 1 mm interval was measured using the NIH Image J processing software and compared with histology.

The fluorescence intensity was evaluated from a total of 277 sites from n=9 EMR specimens collected from n=9 subjects. These intervals were found to be squamous (n=107), intestinal metaplasia (n=66), dysplasia (n=69) and normal gastric mucosa (n=35) on histology. The mean intensity values for dysplasia, intestinal metaplasia, squamous and gastric mucosa were 56.5±, 42.1±, 27.1±, and 24.1±gray levels, respectively. Analysis of variance showed an F-statistic of 13.2 (p=<0.0001). Two-sample t-testing showed the mean intensity value for dysplasia was higher than the mean intensity values for intestinal metaplasia (t=2.2, p<0.05), squamous (t=5.2, p<0.01) and gastric mucosa (t=5.01, p<0.01). The mean intensity value for intestinal metaplasia was higher than the mean intensity values of squamous (t=3.06, p<0.01) and gastric mucosa (t=3.04, p<0.01). There was no statistically significant difference in the mean intensity values between squamous and gastric mucosa (t=0.38, p=0.71).

Selective binding of the novel fluorescence-labeled SNF polypeptide to Barrett's dysplasia was therefore demonstrated over mucosal surface areas on the centimeter scale.

1. A composition comprising a polypeptide consisting essentially of the amino acid sequence set out in SEQ ID NO: 1 (SNFYMPL), a linker sequence and a detectable marker; said detectable marker connected to said polypeptide through said linker, said linker having a net neutral charge, and wherein the presence of said linker results in an increase in detectable binding of said polypeptide sequence to Barrett's esophageal tissue compared to the detectable binding of said polypeptide sequence to Barrett's esophageal tissue in the absence of said linker.

2. The composition of claim 1 wherein the polypeptide consists of the amino acid sequence set out in SEQ ID NO: 1.

3. The composition of claim 1 wherein a terminal amino acid of the linker is lysine.

4. The composition of claim 1 wherein the linker comprises the sequence set out in SEQ ID NO: 2 (GGGSK).

5. The composition of claim 1 wherein the detectable marker is fluorescein isothiocyanate (FITC).

6. A pharmaceutical composition comprising the composition according to claim 1 and a pharmaceutically acceptable excipient.

7. The composition of claim 1, further comprising an additional moiety.

8. The composition of claim 7 wherein the moiety is selected from the group consisting of a chemotherapeutic agent, a thereapeutic agent, a polypeptide, an antibody, a nucleic acid, a small molecule or combinations thereof.

9. A method of detecting a adenocarcinoma cell comprising the step of administering the composition of claim 1 to Barrett's esophageal tissue in an amount effective to detect the adenocarcinoma cell, said composition having a property of preferentially binding to the adenocarcinoma cell relative to a non-cancerous cell.

10. A method of determining effectiveness of a treatment for dysplastic Barrett's esophagus in a human comprising the step of administering the composition of claim 1 to the human in an amount effective to label dysplastic Barrett's esophagus, visualizing a first amount of cells labeled with the composition of claim 1, and comparing the first amount to a previously visualized second amount of cells labeled with the composition of claim 1, wherein a decrease in the first amount cells labeled relative to the previously visualized amount of cells labeled is indicative of effective treatment.

11. The method of claim 9 further comprising obtaining a biopsy of the cell labeled by the composition of claim 1.

12. The method of claim 9 wherein the composition is administered after clinical onset of dysplastic Barrett's esophagus.

13. A kit comprising a pharmaceutical composition of claim 6, instructions for use of the composition and a device for administering said pharmaceutical composition to the patient.


SEQUENCE LISTING (7 sequences)

SEQ ID NO: 1 - protein, 7 aa, Artificial Sequence
  Synthetic peptide
SNFYMPL

SEQ ID NO: 2 - protein, 5 aa, Artificial Sequence
  Synthetic peptide
GGGSK

SEQ ID NO: 3 - protein, 7 aa, Artificial Sequence
  Synthetic peptide
VATQAYL

SEQ ID NO: 4 - protein, 12 aa, Artificial Sequence
  Synthetic peptide
GLKIWSLPPHHG

SEQ ID NO: 5 - protein, 7 aa, Artificial Sequence
  Synthetic peptide
ASYNYDA

SEQ ID NO: 6 - protein, 12 aa, Artificial Sequence
  Synthetic peptide
SNFYMPLGGGSK

SEQ ID NO: 7 - protein, 12 aa, Artificial Sequence
  Synthetic peptide
NLMPYFSGGGSK